(19) United States
(12) Patent Application Publication
(10) Pub. No.: US 2024/0207635 A1
(43) Pub. Date: Jun. 27, 2024

(54) Method Of And System For Treatment Of Musculoskeletal Conditions With A Synergistic Combination Of Therapeutic Modalities

(71) Applicant: Hecker Sports Medicine, LLC, Fort Collins, CO (US)

(72) Inventor: Thomas Marion Hecker, Fort Collins, CO (US)

(73) Assignee: Hecker Sports Medicine, LLC, Fort Collins, CO (US)

(21) Appl. No.: 18/145,508

(22) Filed: Dec. 22, 2022

(51) Int. Cl. A61N 5/06; A61F 7/00; A61H 23/00; A61H 23/02; A61N 5/067

(52) U.S. Cl. CPC A61N 5/0613; A61F 7/007; A61H 23/008; A61H 23/0236; A61H 23/0245; A61N 5/067; A61F 2007/0071; A61H 2201/0207; A61H 2201/0228; A61H 2201/5002; A61N 2005/0659

(57) ABSTRACT

A method of, and system for, treatment of chronic and acute musculoskeletal conditions with therapeutic modalities is provided. The method and system treat an area of a body with a plurality of treatment sessions in which a treatment session thereof includes a synergistic combination of sequentially administered therapeutic modalities administered to the area of the body. The synergistic combination of sequentially administered therapeutic modalities includes laser therapy, capacitive resistive electric transfer therapy, shock wave therapy, and acoustical vibration therapy.

### FIELD OF THE INVENTION

The present invention is directed to a method of, and system for, treatment of musculoskeletal conditions with a combination of therapeutic modalities, and more particularly, to a method of, and system for, treatment of chronic and acute musculoskeletal conditions with a synergistic combination of sequentially administered therapeutic modalities.

### BACKGROUND OF THE INVENTION

Musculoskeletal conditions and disorders, such as chronic and acute musculoskeletal conditions, have been treated in a variety of ways. For example, in some cases, surgery may be performed to treat musculoskeletal conditions and disorders. In other cases, stem cells may be harvested and injected into an area of the body to provide an improved anti-inflammatory effect, to start a healing effect, etc. In some cases, patients may prefer non-surgical solutions for pain or damaged tissue or a patient may not be a candidate for a surgical procedure. Various therapeutic modalities individually are known for attempting to treat musculoskeletal conditions and disorders, such as chronic and acute musculoskeletal conditions, without surgery or for treating such conditions in recovery or rehabilitation following surgical treatments. Non-surgical therapeutic modalities generally refer to the administration of one of thermal, mechanical, electromagnetic, and light energies for therapeutic purposes and generally are used individually by physiotherapists to help a patient achieve therapy goals, such as pain relief or modulation, reducing inflammation, improving circulation, tissue healing, scar tissue remodeling, skin condition treatment, enhancing muscle activation, decreasing unwanted muscular activity, preservation or increasing of strength after injury or surgery, among other things. The term “therapeutic modalities” has been used interchangeably with terms such as “electrophysical agents” or “electrophysical devices” to describe interventions that promote or produce physiological therapeutic effects or devices used to promote or produce such physiological therapeutic effects. A non-limiting list of examples of therapeutic modalities may include, for example, electrical stimulation/iontophoresis, biofeedback, thermotherapy (superficial or deep), cryotherapy, ultrasound/phonophoresis, extracorporeal shockwave therapy (ESWT), laser therapy, magnetic therapy, massage, mechanical traction, non-invasive radio frequency treatment such as capacitive resistive electric transfer therapy (commonly referred to as TECAR therapy, which is an acronym for the Spanish language name Transferencia Eléctrica Capacitiva Resistiva), etc.

### SUMMARY OF THE INVENTION

The present invention recognizes that, while various benefits of each of the known therapeutic modalities individually may be known and/or may have been theorized, the potentially synergistic effects of combinations of therapeutic modalities employed together, and more particularly, the synergistic effects and advantages provided by unique and synergistic combinations of therapeutic modalities employed together in particular sequences and at particular timing during treatment sessions and treatment protocols, have not heretofore been unexplored.

These problems and others are addressed by the present invention, a first exemplary embodiment of which comprises a method of treatment of musculoskeletal conditions, such as chronic and acute musculoskeletal conditions, the method comprising the steps of treating an area (e.g., an affected area) of a body with a plurality of treatment sessions, wherein a treatment session of the plurality of treatment sessions comprises a synergistic combination of sequentially administered therapeutic modalities administered to the area, and wherein the therapeutic modalities comprise laser therapy, capacitive resistive electric transfer therapy, shock wave therapy, and acoustical vibration therapy. The exemplary embodiments can provide a unique and synergistic combination of sequentially administered therapeutic modalities that can improve tissue healing or tissue regeneration by stimulating the body to achieve a more effective healing response, reduce or eliminate pain, improve recovery time, improve post-surgery regeneration, among other things, for a patient as compared to the administration of known therapeutic modalities individually.

For example, the exemplary embodiments can provide a unique and synergistic combination of sequentially administered therapeutic modalities that can allow for a high level of success in recovery of musculoskeletal disorders, for example, by providing for a high level of stimulation to the stem cells in the region of an injury that promote healing. More particularly, the exemplary embodiments can stimulate endogenous stem cell activity, release nitric oxide to produce an accelerated healing effect, pain relief effect, vascular effect, and/or mitochondrial effect, and improve stabilization of surrounding muscular structures involved in either the chronic or acute injury cycle during the treatment sequences and sessions. Moreover, the synergistic combination of sequentially administered therapeutic modalities according to the exemplary embodiments can, among other things, provide improved pain relief during the early phases of the treatment process and throughout the duration of the treatments sessions, as well as improve function, range of motion/movement, and strength throughout the duration of the treatment sessions, thereby enabling a patient to stay active, for example, with no restrictions or with minor/minimal restrictions on activities.

In some examples, the therapeutic modalities can comprise at least three of laser therapy, capacitive resistive electric transfer therapy, shock wave therapy, and acoustical vibration therapy. In other examples, the therapeutic modalities can comprise laser therapy, capacitive resistive electric transfer therapy, shock wave therapy, and acoustical vibration therapy.

In still other examples, the therapeutic modalities can comprise laser therapy followed by at least one of capacitive resistive electric transfer therapy, shock wave therapy, and acoustical vibration therapy. For example, a treatment session can comprise administering laser therapy to the area followed by administering at least one of capacitive resistive electric transfer therapy, shock wave therapy, and acoustical vibration therapy to the area. In other examples, a treatment session can comprise administering the laser therapy to the area followed by administering at least two of the capacitive resistive electric transfer therapy, the shock wave therapy, and the acoustical vibration therapy to the area. In a particular example, a treatment session can comprise administering the laser therapy to the area followed by administering at least one of capacitive resistive electric transfer therapy and shock wave therapy to the area, and further followed by administering acoustical vibration therapy to the area. In yet another example, a treatment session can comprise administering laser therapy to the area followed by administering capacitive resistive electric transfer therapy to the area, followed by administering shock wave therapy to the area, and followed by administering acoustical vibration therapy to the area.

In another particular example, for a first predetermined number of treatment sessions of a plurality of treatment sessions, the step of treating can comprise administering laser therapy to the area followed by administering capacitive resistive electric transfer therapy to the area. In other examples, for the first predetermined number of treatment sessions of the plurality of treatment sessions, the step of treating can further comprise administering acoustical vibration therapy to the area following the administration of the capacitive resistive electric transfer therapy. In another example, for the first predetermined number of treatment sessions of the plurality of treatment sessions, the step of treating can further comprise administering acoustical vibration therapy to the area following the administration of the capacitive resistive electric transfer therapy every other sequential treatment session (e.g., only every other sequential treatment session).

In still other examples, for a second predetermined number of treatment sessions of the plurality of treatment sessions, the step of treating can further comprise administering laser therapy to the area followed by administering shock wave therapy to the area. The second predetermined number of treatment sessions can follow a completion of at least one of the first predetermined number of treatment sessions, or in another example, the second predetermined number of treatment sessions can follow a completion of all of the first predetermined number of treatment sessions. In a further example, for the second predetermined number of treatment sessions of the plurality of treatment sessions, the step of treating can further comprise administering acoustical vibration therapy to the area following the administration of the shock wave therapy. In another example, for the second predetermined number of treatment sessions of the plurality of treatment sessions, the step of treating can further comprise administering acoustical vibration therapy to the area following the administration of the shock wave therapy every other sequential treatment session (e.g., only every other sequential treatment session).

With regard to laser therapy, an example of this therapeutic modality can include treating at least a portion of the area using a near infrared laser therapy treatment device. In a preferred embodiment, the laser therapy can be administered using a laser treatment device supplying a 1064 nm laser to the portion of the area. The laser treatment device can comprise, for example, a 1 W to 12 W laser. In a preferred example, the laser treatment device can be operated at a maximum power density for the laser treatment device throughout the duration (e.g., the entire duration) of supplying light energy to the area during a treatment session. For example, the laser treatment device can be operated at a maximum power density of 450 mW/cm² throughout the entire process of administering the laser therapy to the area of the body during a treatment session.

As part of the synergistic combination of sequentially administered therapeutic modalities according to the exemplary embodiments, the treating of the area using the laser therapy can have a strong effect on mitochondria stimulation and can increase the level of mitochondrial activity within the endogenous stem cells being treated. This higher level of mitochondrial activity can enhance cellular function and production of proteins for recovery of musculoskeletal conditions. Upon stimulating the mitochondrial effect, the exemplary method can promote the propagation of the mitochondrial effect through the structure (e.g., throughout the whole structure), thereby maximizing the mitochondrial effect. For example, the treatment can open cell wall function and increase transmission of nutrients into cells and waste out of cells. The treatment also can produce an ionic effect that increases blood flow and increases pain relief and healing.

With regard to the capacitive resistive electric transfer therapy, an example of this therapeutic modality can include treating at least a portion of the area using a TECAR therapy device. TECAR therapy uses endogenous heat production to treat musculoskeletal conditions, such as musculoskeletal traumas and pathologies. TECAR therapy uses heat, generated by the movement of electric charges, to stimulate reparative processes and to provide an anti-inflammatory and analgesic effect. TECAR therapy can be used in different ways on the treated area using two different transfer methods including a capacitive mode (e.g., for superficial heating) and a resistive mode (e.g., for deeper heating). In a preferred example, the TECAR therapy includes treating at least a portion of the area of the body using the capacitive mode followed by treating the portion of the area using the resistive mode. An example TECAR therapy device can be configured to supply capacitive and resistive radio frequency currents from 300 kHz to 1 MHz. As part of the synergistic combination of sequentially administered therapeutic modalities according to the exemplary embodiments, the spectrum of currents of the capacitive resistive electric transfer therapy can allow for a biological, cell wall, analgesic, and diathermy effect to the treated tissue. A primary endogenous effect can be cell wall permeability and increased blood volume. In the examples, the synergistic combination of sequentially administered laser treatment followed by capacitive resistive electric transfer therapy can, among other things, provide improved pain relief during the early phases of the treatment process and throughout the duration of the treatments sessions.

With regard to the shock wave therapy, an example of this therapeutic modality can include treating at least a portion of the area using, for example, a piezoelectric shock wave device that transforms the mechanical energy of soundwaves into biochemical signals in target tissue. This acoustic compression can trigger a cascade of cellular and molecular responses through a process called mechanotransduction. Piezoelectric shock wave is a focused shockwave that creates a deep cellular effect to tissue with collagen and M2 macrophage transition. This is a mechanical transduction treatment that allows for the transition of tissue, in instances of chronic or acute musculoskeletal conditions, to progress from scarred state to natural physiological properties. Shock wave therapy can be used, for example, on any area of the body having, for example, collagen, including tendons. An example piezoelectric shock wave device employed according to an exemplary embodiment of the invention can have a frequency of from 1 Hz to 12 Hz. While shock wave therapy may be uncomfortable to many patients, as part of the synergistic combination of sequentially administered therapeutic modalities according to the exemplary embodiments, over a number of treatment sessions, the combination of the shock wave therapy with the other therapeutic modalities can improve the state of the treated tissue such that a patient can better tolerate the treatment, thereby enabling a deeper remodeling effect of the tissue over the course of the treatment protocol. In an example protocol, a power and a frequency of the piezoelectric shock wave device can be increased (e.g., incrementally increased, gradually increased, periodically increased, etc.) with each successive treatment session of a plurality of treatment sessions. As part of the synergistic combination of sequentially administered therapeutic modalities according to the exemplary embodiments, over a number of treatment sessions, the shock wave therapy also can provide an anti-inflammatory effect. In the examples, the synergistic combination of sequentially administered laser treatment and capacitive resistive electric transfer therapy can, among other things, prepare the tissue to be more responsive to the subsequent shock wave therapy.

With regard to the acoustical vibration therapy, an example of this therapeutic modality can include treating at least a portion of the area of the body using an acoustical vibration device configured to supply a plurality of frequencies and a plurality of amplitudes of mechanical vibration to the portion of the area. For example, the acoustical vibration device can be configured to supply acoustical vibration having a frequency of 0-300 Hz. The acoustical vibration device can be configured to provide a range of amplitudes of acoustical vibration, such as a range of amplitude settings of 1-12, with 1 being the lowest amplitude setting and 12 being the highest amplitude setting. In an example protocol, a frequency and an amplitude of the acoustical vibration supplied by the acoustical vibration device can be increased (e.g., incrementally increased, gradually increased, periodically increased, etc.) with each successive treatment session of the plurality of treatment sessions over the course of the treatment protocol. In an example treatment session, a frequency and an amplitude of the acoustical vibration supplied by the acoustical vibration device can progress sequentially from eliciting a slow twitch response, an intermediate twitch response, and a fast twitch response. As part of the synergistic combination of sequentially administered therapeutic modalities according to the exemplary embodiments, the stimulation provided by the acoustical vibration therapy can have a profound effect on muscle nerve fibers and Pacinian corpuscles to cause muscle transition of fiber type, i.e., strengthening as well as stimulation of growth hormone production and reduction of neuromuscular tightness and improvement of muscular pliability. Moreover, as part of the synergistic combination of sequentially administered therapeutic modalities according to the exemplary embodiments, the acoustical vibration therapy can reduce tightness (e.g., muscle tightness), create a growth factor effect (e.g., increased hormones), improve flexibility and strength of the muscle tissue, among other things.

In the examples, the synergistic combination of sequentially administered laser treatment and capacitive resistive electric transfer therapy can, among other things, prepare the tissue to be more responsive to the acoustical vibration therapy, thereby achieving improved function, range of motion/movement, and strength throughout the duration of the treatment sessions, thereby enabling a patient to stay active, for example, with no restrictions or with minor/minimal restrictions on activities.

In an example, the overall number of treatment sessions can be equal to or greater than 10 treatments, and more particularly, greater than 14 treatments, and even more particularly, greater than eighteen treatment sessions. In a particular example, the overall number of treatment sessions can include a number of first treatment sessions and a number of second treatment sessions, wherein the therapeutic modalities administered in at least one of the first treatment sessions is different from the therapeutic modalities administered in at least one of the second treatment sessions. The number of the first treatment sessions and/or the second treatment sessions can be equal to or greater than 5 treatment sessions, and more particularly, greater than 7 treatment sessions, and even more particularly, greater than 9 treatment sessions. In some examples, the number of first and/or second treatment sessions can range from 5-7 sessions, 5-9 sessions, etc.

Another exemplary embodiment is directed to a method of treatment of musculoskeletal conditions, the method comprising the steps of treating an area of a body with a plurality of treatment sessions, wherein each treatment session of the plurality of treatment sessions comprises a synergistic combination of sequentially administered therapeutic modalities administered to the area, wherein, in a first subset of the plurality of treatment sessions, the therapeutic modalities comprise laser therapy followed by at least one of capacitive resistive electric transfer therapy, shock wave therapy, and acoustical vibration therapy, wherein, in a second subset of the plurality of treatment sessions, the therapeutic modalities comprise laser therapy followed by at least one of capacitive resistive electric transfer therapy, shock wave therapy, and acoustical vibration therapy, and wherein the therapeutic modalities of the first subset are different from the therapeutic modalities of the second subset.

Another exemplary embodiment is directed to a system for treatment of musculoskeletal conditions, the system comprising a laser therapy device, a capacitive resistive electric transfer therapy device, a shock wave therapy device, and an acoustical vibration therapy device, wherein the laser therapy device, the capacitive resistive electric transfer therapy device, the shock wave therapy device, and the acoustical vibration therapy device are configured to treat an area of a body with a plurality of treatment sessions, wherein each treatment session of the plurality of treatment sessions comprises sequentially administering therapeutic modalities to the area using at least two of the laser therapy device, the capacitive resistive electric transfer therapy device, the shock wave therapy device, and the acoustical vibration therapy device.

For purposes of this disclosure, musculoskeletal conditions, such as chronic and acute musculoskeletal conditions, can include, among other things, Achilles tendon injuries, foot pain, ankle pain, stress fractures, plantar fasciitis, flat feet and fallen arches, joint injury, heel spurs, ligament injury, strains and tears, etc. Such injuries may result from repetitive overuse or overload of the affected area, or from stress, impact, or overload during activity or use of the affected area, among other things. The “area of the body” or the “affected area” can include bodily tissue or structures, such as muscle tissue, tendons, ligaments, bone structures, nerve tissue, cardiovascular tissue, etc. For example, the affected area can include all or any part of a foot, ankle, knee, hip, hand, wrist, elbow, shoulder, neck, back, etc.

According to the exemplary embodiments, the present invention can provide a unique and synergistic combination of sequentially administered therapeutic modalities that can improve tissue healing or tissue regeneration by stimulating the body to achieve a more effective healing response, reduce or eliminate pain, improve recovery time, improve post-surgery regeneration, among other things, for a patient as compared to the administration of known therapeutic modalities individually. The exemplary embodiments can provide a unique and synergistic combination of sequentially administered therapeutic modalities that can allow for a high level of success in recovery of musculoskeletal disorders, for example, by providing for a high level of stimulation to the stem cells in the region of an injury that promote healing. More particularly, the exemplary embodiments can stimulate endogenous stem cell activity, produce accelerated healing of an area, or areas, of the body, such as injured structures, and improve stabilization of surrounding muscular structures involved in either the chronic or acute injury cycle during the treatment sequences and sessions. Moreover, the synergistic combination of sequentially administered therapeutic modalities according to the exemplary embodiments can, among other things, improve function, range of motion/movement, and strength, as well as reduce pain, throughout the duration of the treatment sessions, thereby enabling a patient to stay active, for example, with no restrictions or with minor/minimal restrictions on activities.

Other features and advantages of the present invention will become apparent to those skilled in the art upon review of the following detailed description and drawings.

### BRIEF DESCRIPTION OF THE DRAWINGS

These and other aspects and features of embodiments of the present invention will be better understood after a reading of the following detailed description, together with the attached drawings, wherein:

FIG. 1 is a schematic view illustrating various exemplary options for administering various therapeutic modalities for treatment of musculoskeletal conditions according to an exemplary embodiment of the invention.

FIG. 2 is a schematic view of a system for administering various therapeutic modalities for treatment of musculoskeletal conditions. according to an exemplary embodiment of the invention.

FIGS. 3-5 are schematic views illustrating various exemplary methods of administering various therapeutic modalities for treatment of musculoskeletal conditions according to an exemplary embodiment of the invention.

FIGS. 6-14 are schematic views illustrating various exemplary sequences of treatment sessions for administering various therapeutic modalities for treatment of musculoskeletal conditions according to an exemplary embodiment of the invention.

### DETAILED DESCRIPTION OF THE EXEMPLARY EMBODIMENTS OF THE INVENTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

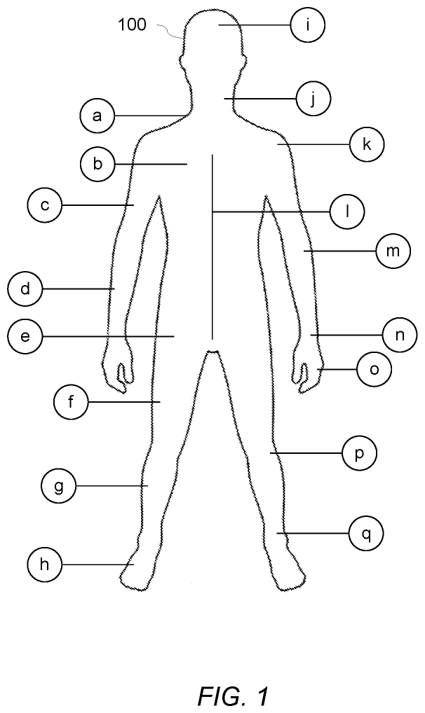

With reference to FIGS. 1-14, exemplary embodiments of a method of, and system for, treatment of chronic and acute musculoskeletal conditions with a synergistic combination of sequentially administered therapeutic modalities will now be described.

FIG. 1 illustrates various exemplary options for administering various therapeutic modalities to an area (e.g., an affected area a-q) of a body 100 for treatment of musculoskeletal conditions. The affected area (e.g., a-q) can include all or any part of a foot, ankle, knee, hip, hand, wrist, elbow, shoulder, neck, back, etc., among other areas, and can include bodily tissue and/or structures, such as muscle tissue, tendons, ligaments, bone structures, etc.

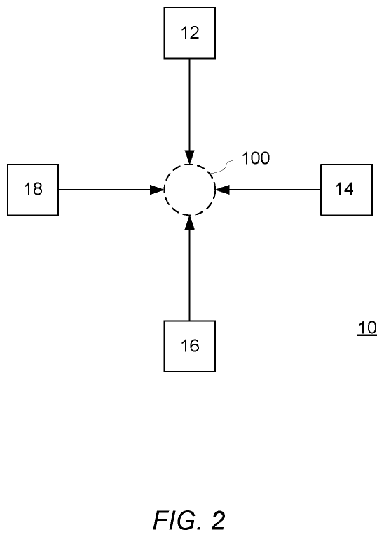

As illustrated n FIG. 2, a system 10 for treatment of musculoskeletal conditions, such as chronic and acute musculoskeletal conditions, and particularly, for treating an area of a body 100 with a plurality of treatment sessions, can include a plurality of electrophysical devices, each being configured to administer a therapeutic modality to the area.

The system 10 can include a laser therapy device 12, such as a near infrared laser therapy device. In an example, the laser therapy device can be a 1064 nm laser. The laser treatment device can comprise, for example, a 1 W to 12 W laser. In an example, the laser treatment device can have a maximum power density of 450 mW/cm². In an example, the laser treatment device can have a 2″ aperture. An example laser photobiomodulation light therapy product suitable for administering laser therapy to the affected area is the CytonPro-5000M manufactured by CytonSys, Inc., Austin, TX.

The system 10 can include a capacitive resistive electric transfer therapy device 14, such as a TECAR therapy device that is configured to operate in a capacitive mode and a resistive mode. An example TECAR therapy device can be configured to supply capacitive and resistive radio frequency currents from 300 kHz to 1 MHz using capacitive and resistive electrodes con figured for transdermal delivery. An example TECAR therapy product suitable for administering capacitive resistive electric transfer therapy to the affected area is the DiaCaRe 7000 manufactured by GLOBUS USA, North Miami Beach, FL.

The system 10 can include a shock wave therapy device 16, such as a piezoelectric shock wave device. An example piezoelectric shock wave device can have a frequency of from 1 Hz to 12 Hz. An example piezoelectric shock wave product suitable for administering shock wave therapy to the affected area is the PiezoWave2T manufactured by ELvation Medical LLC, Alpharetta, GA.

The system 10 can include an acoustical vibration therapy device 18 configured to supply a plurality of frequencies and a plurality of amplitudes of mechanical vibration to the portion of the area. For example, the acoustical vibration device can be configured to supply acoustical vibration having a frequency of 0-300 Hz. The acoustical vibration device can be configured to provide a range of amplitudes of acoustical vibration, such as a range of amplitude settings of 1-12, with 1 being the lowest amplitude setting and 12 being the highest amplitude setting. An example acoustical vibration therapy product suitable for administering acoustical vibration therapy to the affected area is the VISS® 1 EVOLUTION AND VISS® MYOMODULATOR DEVICES manufactured by VISSMAN®, Rome, Italy.

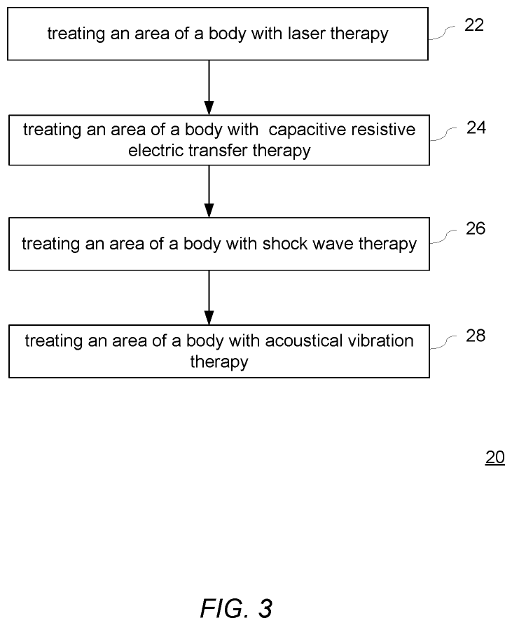

As illustrated in FIG. 3, a method of treatment of musculoskeletal conditions, such as chronic and acute musculoskeletal conditions, and particularly, of treating an area of a body can include a treatment session 20 of a plurality of treatment session. The treatment session 20 can include the steps of treating an area of a body with laser therapy 22, treating an area of a body with capacitive resistive electric transfer therapy 24, treating an area of a body with shock wave therapy 26, and treating an area of a body with acoustical vibration therapy 28.

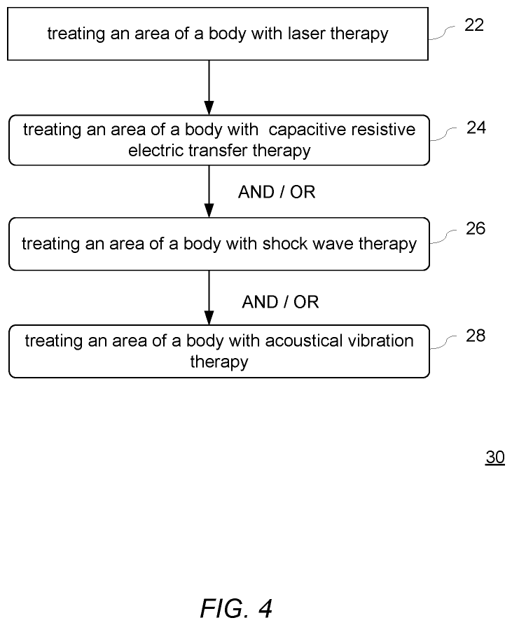

As illustrated in FIG. 4, an example treatment session 30 of a plurality of treatment session of a method of treatment of musculoskeletal conditions can include the sequential steps of treating an area of a body with laser therapy 22 followed by one or more of treating an area of a body with capacitive resistive electric transfer therapy 24, treating an area of a body with shock wave therapy 26, and treating an area of a body with acoustical vibration therapy 28.

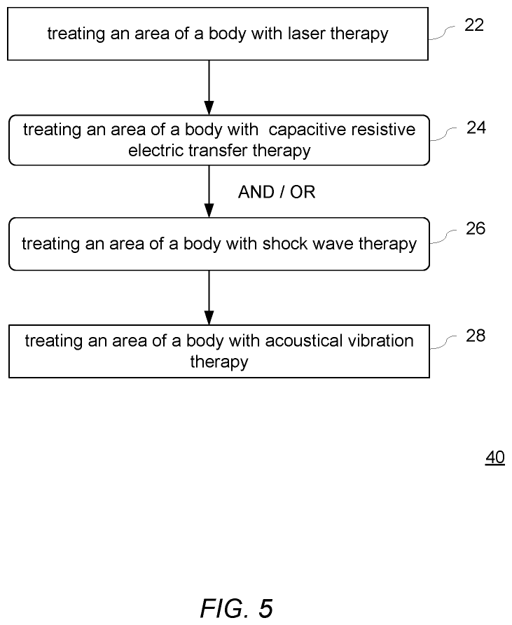

As illustrated in FIG. 5, an example treatment session 40 of a plurality of treatment session of a method of treatment of musculoskeletal conditions can include the sequential steps of treating an area of a body with laser therapy 22 followed by one or more of treating an area of a body with capacitive resistive electric transfer therapy 24 and treating an area of a body with shock wave therapy 26, and followed by treating an area of a body with acoustical vibration therapy 28.

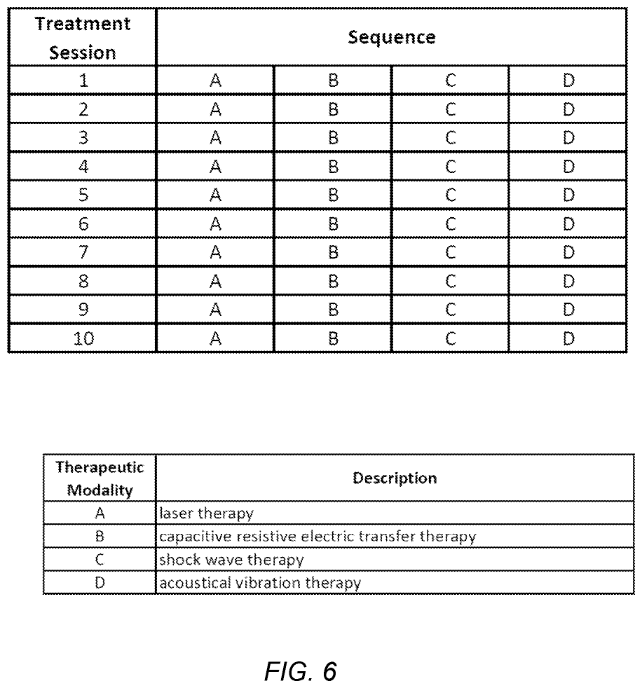

As illustrated in FIG. 6, an example sequence or protocol of treatment sessions of a method of treatment of musculoskeletal conditions can include the sequential steps of treating an area of a body with laser therapy 22, treating an area of a body with capacitive resistive electric transfer therapy 24, treating an area of a body with shock wave therapy 26, and treating an area of a body with acoustical vibration therapy 28 during each treatment session. The time needed to complete a full sequence of each of the therapeutic modalities in each treatment session may limit the suitability of this example for some patients.

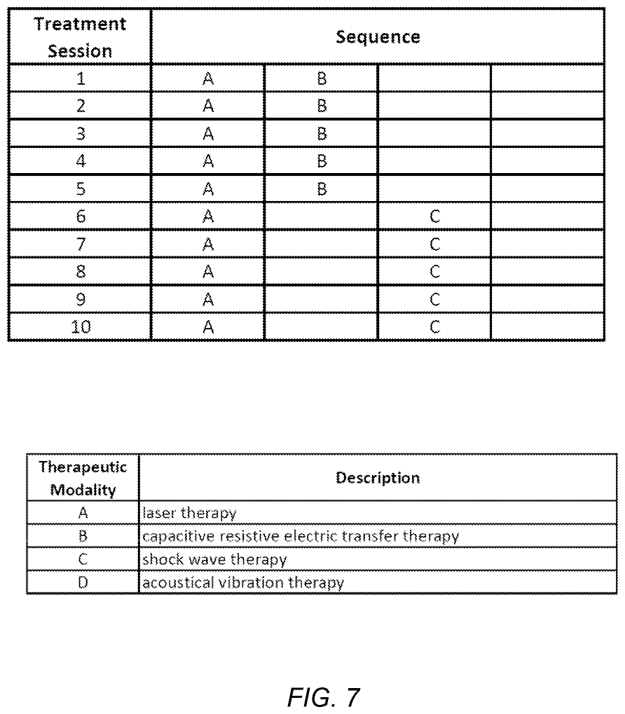

As illustrated in FIG. 7, in an example sequence or protocol of treatment sessions, a first predetermined number of treatment sessions, such as five (5) sessions, can include administering laser therapy to the area followed by administering capacitive resistive electric transfer therapy to the area. A second predetermined number of treatment sessions, such as five (5) sessions, can include administering laser therapy to the area followed by administering shock wave therapy to the area. The second predetermined number of treatment sessions can follow a completion of all of the first predetermined number of treatment sessions, as shown in the example, or in other examples, the second predetermined number of treatment sessions can follow a completion of at least one of the first predetermined number of treatment sessions.

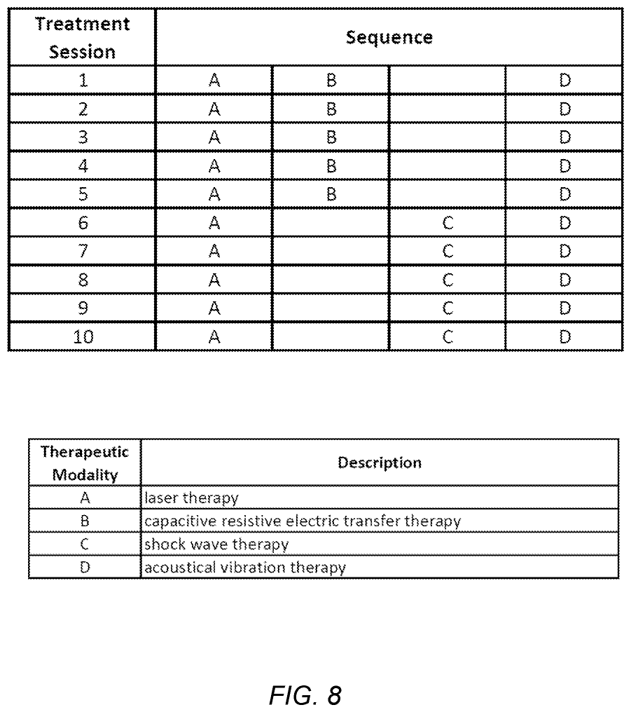

As illustrated in FIG. 8, in another example sequence or protocol of treatment sessions, acoustical vibration therapy can be administered to the area following each of the administration of the capacitive resistive electric transfer therapy and the administration of the shock wave therapy.

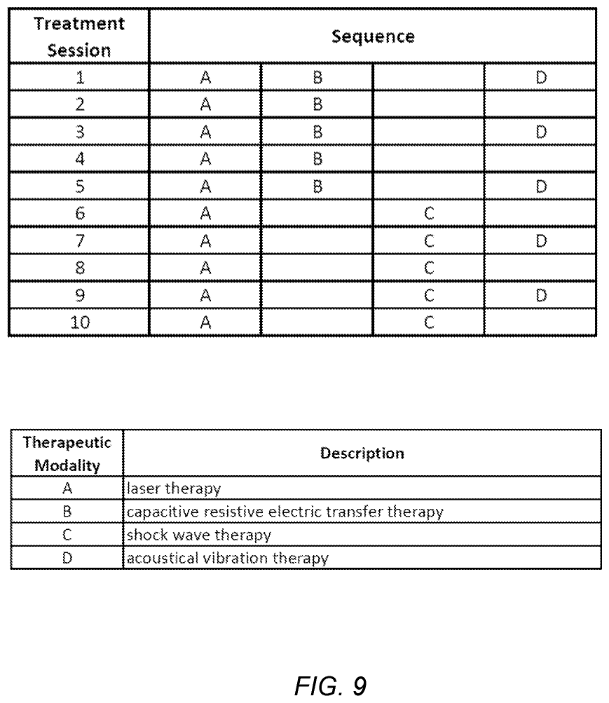

As illustrated in FIG. 9, in another example sequence or protocol of treatment sessions, acoustical vibration therapy can be administered to the area following the administration of the capacitive resistive electric transfer therapy every other sequential treatment session (e.g., only every other sequential treatment session) and/or following the administration of the shock wave therapy every other sequential treatment session (e.g., only every other sequential treatment session).

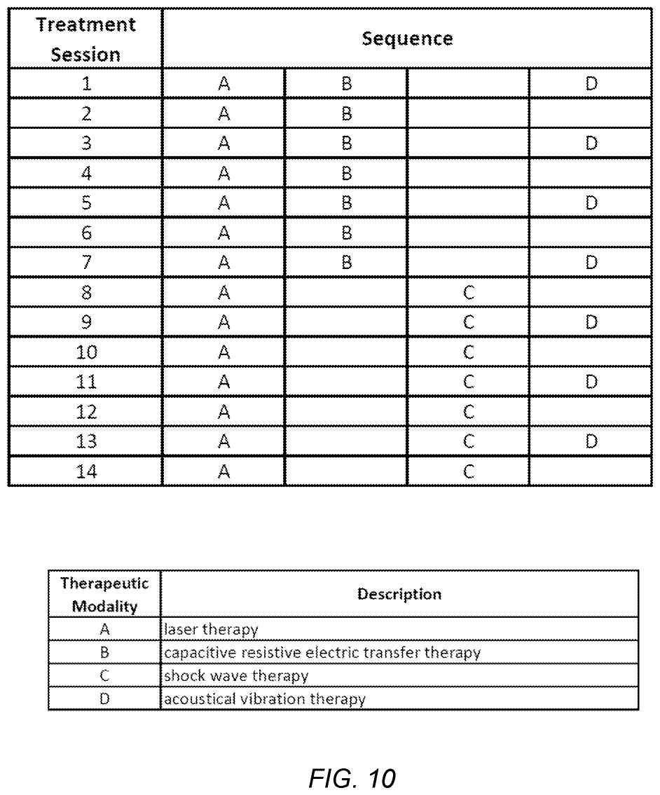

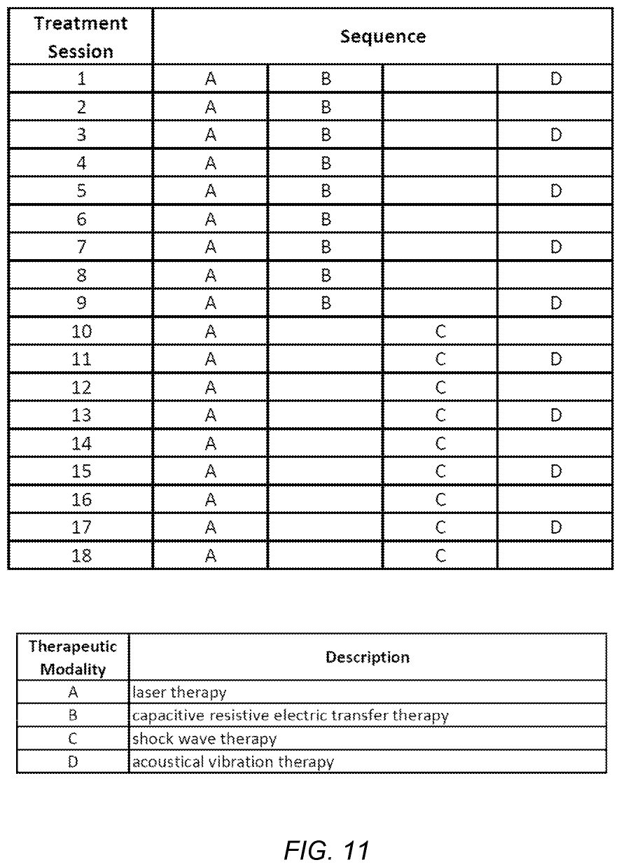

As illustrated in FIGS. 10 and 11, in other example sequences or protocols of treatment sessions, the first and second predetermined number of treatment sessions each can include seven (7) sessions, nine (9) sessions, etc., for a total of fourteen (14) sessions, eighteen (18) sessions, etc. The number of the first treatment sessions can be the same as or different from the number of second treatment sessions.

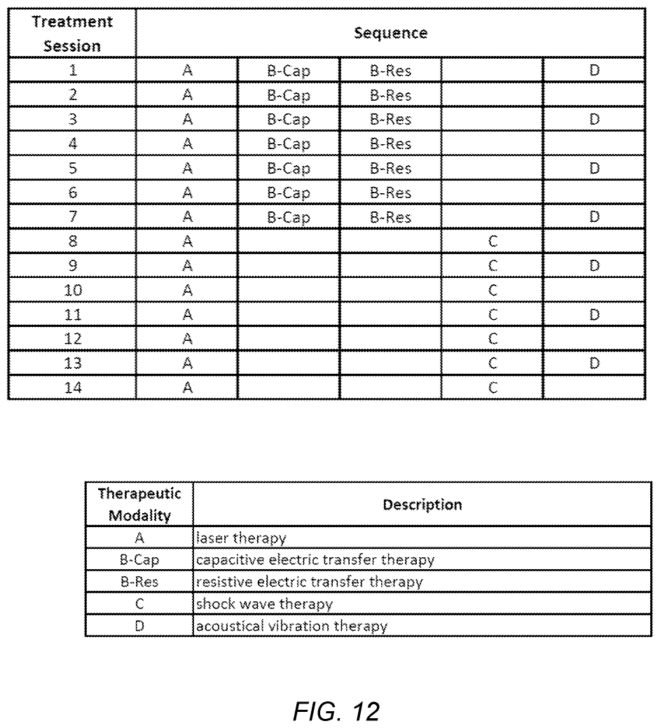

As illustrated in FIG. 12, the capacitive resistive electric transfer therapy can include treating at least a portion of the area, for example using a TECAR therapy device, in a capacitive mode followed by treating at least a portion of the area in a resistive mode. The capacitive resistive electric transfer therapy can be administered over a spectrum of currents during each of the capacitive and resistive modes.

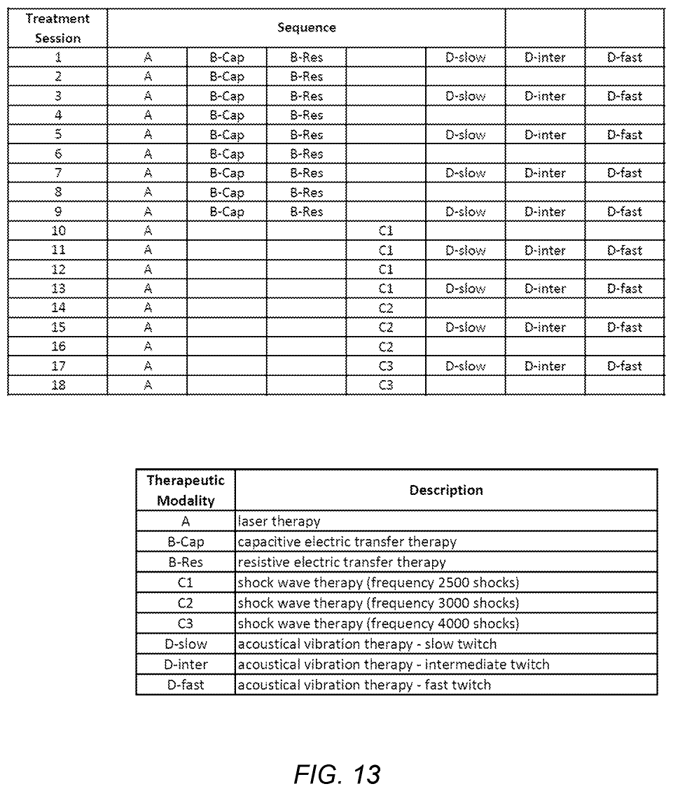

As illustrated in the example of FIG. 13, the shock wave therapy can include treating at least a portion of the area using, for example, a piezoelectric shock wave device. An example piezoelectric shock wave device employed according to an exemplary embodiment of the invention can have a frequency of from 1 Hz to 12 Hz. In an example, a frequency of the device can be adjusted to administer a predetermined initial number of shockwaves. The number of shockwaves can then be increased (e.g., incrementally increased, gradually increased, etc.) over the course of subsequent sessions, such as each successive session, every other session, every few sessions, etc. For example, in FIG. 13, the number of shockwaves initially administered can be 2500 shock waves, followed by subsequent increases in the number of shock waves during other sessions to 3000 shock waves, and up to 4000 shockwaves (e.g., a maximum number of shockwaves). The increase in the number of shockwaves, and the rate of increase, can be based on a particular patient, and particularly, the number of shockwaves that the particular patient can tolerate. The power density of the device similarly can be increased (e.g., incrementally increased, gradually increased, etc.) over the course of subsequent sessions, such as each successive session, every other session, every few sessions, etc.

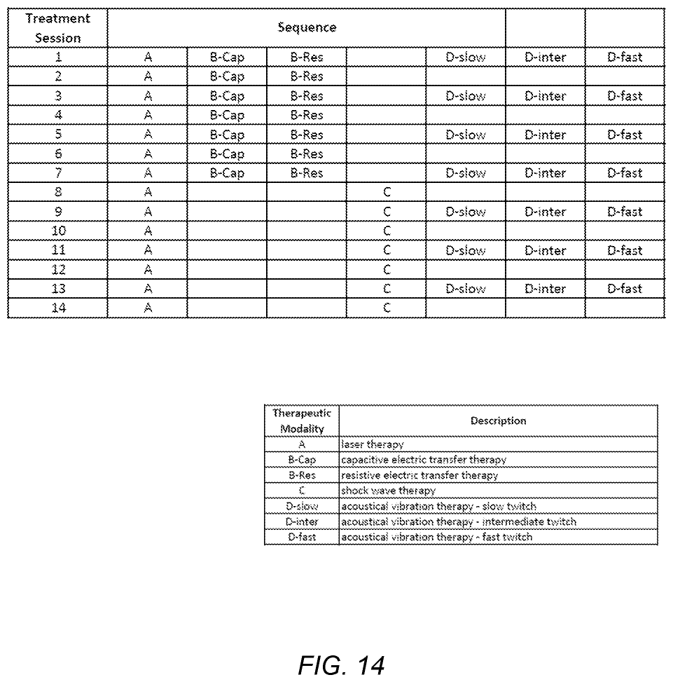

As illustrated in the example of FIG. 14, the acoustical vibration therapy can include treating at least a portion of the area using, for example, an acoustical vibration device configured to supply a plurality of frequencies and amplitudes of mechanical vibration to the area. The acoustical vibration device can be configured to supply acoustical vibration having a frequency of 0-300 Hz. The acoustical vibration device can be configured to provide a range of amplitudes of acoustical vibration, such as a range of amplitude settings of 1-12, with 1 being the lowest amplitude setting and 12 being the highest amplitude setting. A frequency and an amplitude of the acoustical vibration can be increased (e.g., incrementally increased, gradually increased, periodically increased, etc.) with each successive treatment session over the course of the treatment protocol. In the illustrated example sequence, a frequency and an amplitude of the acoustical vibration supplied by the acoustical vibration device can be administered to progress sequentially from eliciting a slow twitch response, an intermediate twitch response, and a fast twitch response during each session.

With reference to the examples illustrated in FIGS. 6-14, the overall number of treatment sessions can range, for example, from 10 to 18 treatments. The number of treatment sessions can be modified or determined based on the particular patient and/or musculoskeletal condition. As shown in the examples, the overall number of treatment sessions can include a number of first treatment sessions and a number of second treatment sessions, wherein the therapeutic modalities administered in one or more of the first treatment sessions is different from the therapeutic modalities administered in one or more of the second treatment sessions. In some examples, the number of first and/or second treatment sessions can range from 5-7 sessions, 5-9 sessions, etc.

### Other Combinations or Variations of the Treatment Sessions and a Variations of the Treatment

In some examples, multiple treatment sessions can be performed on the same day, or treatment sessions can be performed on different days. For example, 2-3 treatment sessions can be performed in a single day. The overall number of treatment sessions can be performed over one or more days, weeks, or months. The treatment sessions can be conducted over each of a number of successive days or spaced out (e.g., equally spaced or unequally spaced) over a larger period of time with one or more days separating one or more of the treatment sessions. For example, one or more treatment sessions can be performed once per week, twice per week, etc.

In a preferred example, the treatment sessions can be performed over a more condensed schedule in order to stimulate and/or accelerate the mitochondrial effect and promote the propagation of the mitochondrial effect through the structure (e.g., throughout the whole structure). However, not all of the treatment sessions need to be performed over a condensed schedule to take advantage of the mitochondrial effects. In an example, an initial set of treatment sessions can be condensed into a shorter period of time, such as 3-4 initial treatment sessions, in order to stimulate and/or accelerate the mitochondrial effect and promote the propagation of the mitochondrial effect through the structure (e.g., throughout the whole structure). In this example, the initial 3-4 treatment sessions can performed, for example, at a minimum once every 7-10 days, and preferably more than once every 7-10 days, to stimulate the mitochondrial effect. Once the mitochondrial effect has been established, the remaining treatment sessions can be spread out over a larger period of time (e.g., greater than 7-10 days), if desired, and without negatively affecting the propagation of the mitochondrial effect throughout the structure and/or the improved effects of the other therapeutic modalities.

With regard to the synergistic combination of sequentially administered laser therapy followed by capacitive resistive electric transfer therapy, there generally is no limit (e.g., safety limit) or negative consequence to how many treatments can be performed in a given period of time (e.g., condensed period of time), other than constraints associated with the time to administer the modalities. For example, when treating an affected area, each therapeutic modality may be administered over or along a course of the affected area, and in some instances, an area surrounding the affected area (e.g., bilateral administration of each therapeutic modality at the affected area). The administration of each therapeutic modality will require a period of time to complete. For example, in a case of treating an affected area such as an Achilles tendon, the laser therapy may be administered over a course of the Achilles tendon and for a period of time for each portion of the tendon, such as 2-3 minutes on the heal, 2-3 minutes on the muscle junction, and 2-3 minutes on the head of the gastrocnemius muscle. By utilizing a near infrared laser therapy treatment device, such as a 1064 nm laser, and more particularly, a low heat optical laser, concerns regarding heat exposure to the patient can be minimized or avoided. As a result, the affected area can be treated with the laser therapy for longer periods of time and may be limited only to external time constraints, such as cost, availability of patient, technician, or equipment, etc.

On the other hand, a time and amount of the synergistic combination of sequentially administered laser therapy followed by shock wave therapy that may be administered to a patient may be limited to the patient, and particularly, to how much of the shock wave therapy the patient can tolerate. For example, in some instances, the shock wave therapy may be limited to a maximum of 8000 shocks per day. In order to administer this amount of shocks, as explained previously, the number of shockwaves can be initially administered at a lower amount and then increased (e.g., incrementally increased, gradually increased, etc.) over the course of subsequent sessions, such as each successive session, every other session, every few sessions, etc., until the patient is capable of tolerated the desired number of shockwaves. Additionally or alternatively, the treatment of the shock wave therapy can be divided into sub-sessions throughout the day, such as 4000 shock waves in the morning and 4000 shock waves in the afternoon/evening to achieve the desired number of shocks per day, such as a maximum 8000 shocks per day.

The exemplary embodiments are described with reference to treatment of areas of the body or affected areas, such as muscle tissue, tendons, ligaments, bone structures, etc. In other examples, one or more of the sequentially administering therapeutic modalities can be modified, or eliminated, to adapt the method of, and system for, treating conditions of the body to a particular area or part of the body, such as nerve tissue, spinal cord tissue, connective tissue of the brain, cardiovascular tissue, etc. For example, the shock wave therapy can be modified, or removed from the protocol altogether, for one or more of such applications.

The present invention has been described herein in terms of several preferred embodiments. However, modifications and additions to these embodiments will become apparent to those of ordinary skill in the art upon a reading of the foregoing description. It is intended that all such modifications and additions comprise a part of the present invention to the extent that they fall within the scope of the several claims appended hereto.

What is claimed is:

1. A method of treatment of musculoskeletal conditions, the method comprising the steps of:
treating an area of a body with a plurality of treatment sessions,
wherein a treatment session of the plurality of treatment sessions comprises a synergistic combination of sequentially administered therapeutic modalities administered to the area, and
wherein the therapeutic modalities comprise at least three of laser therapy, capacitive resistive electric transfer therapy, shock wave therapy, and acoustical vibration therapy.

2. The method of claim 1, wherein the therapeutic modalities comprise the laser therapy, the capacitive resistive electric transfer therapy, the shock wave therapy, and the acoustical vibration therapy.

3. The method of claim 1, wherein the treatment session comprises administering the laser therapy to the area followed by administering at least one of the capacitive resistive electric transfer therapy, the shock wave therapy, and the acoustical vibration therapy to the area.

4. The method of claim 1, wherein the treatment session comprises administering the laser therapy to the area followed by administering at least two of the capacitive resistive electric transfer therapy, the shock wave therapy, and the acoustical vibration therapy to the area.

5. The method of claim 1, wherein the treatment session comprises administering the laser therapy to the area followed by administering at least one of the capacitive resistive electric transfer therapy and the shock wave therapy to the area, and followed by administering the acoustical vibration therapy to the area.

6. The method of claim 1, wherein the treatment session comprises administering the laser therapy to the area followed by administering the capacitive resistive electric transfer therapy to the area, followed by administering the shock wave therapy to the area, and followed by administering the acoustical vibration therapy to the area.

7. The method of claim 1, wherein, for a first predetermined number of treatment sessions of the plurality of treatment sessions, the step of treating comprises:
administering the laser therapy to the area followed by administering the capacitive resistive electric transfer therapy to the area.

8. The method of claim 7, wherein, for the first predetermined number of treatment sessions of the plurality of treatment sessions, the step of treating further comprises:
administering the acoustical vibration therapy to the area following the capacitive resistive electric transfer therapy.

9. The method of claim 7, wherein, for the first predetermined number of treatment sessions of the plurality of treatment sessions, the step of treating further comprises:
administering the acoustical vibration therapy to the area following the capacitive resistive electric transfer therapy every other sequential treatment session.

10. The method of claim 7, wherein, for a second predetermined number of treatment sessions of the plurality of treatment sessions, the step of treating further comprises:
administering the laser therapy to the area followed by administering the shock wave therapy to the area.

11. The method of claim 10, wherein the second predetermined number of treatment sessions follows a completion of at least one of the first predetermined number of treatment sessions.

12. The method of claim 10, wherein the second predetermined number of treatment sessions follows a completion of all of the first predetermined number of treatment sessions.

13. The method of claim 10, wherein, for the second predetermined number of treatment sessions of the plurality of treatment sessions, the step of treating further comprises:
administering the acoustical vibration therapy to the area following the shock wave therapy.

14. The method of claim 10, wherein, for the second predetermined number of treatment sessions of the plurality of treatment sessions, the step of treating further comprises:
administering the acoustical vibration therapy to the area following the shock wave therapy every other sequential treatment session.

15. The method of claim 1, wherein the laser therapy includes treating at least a portion of the area using a near infrared laser therapy treatment device.

16. The method of claim 1, wherein the laser treatment device supplies a 1064 nm laser to the portion of the area.

17. The method of claim 1, wherein the laser treatment device comprises a 1 w to 12 w laser.

18. The method of claim 1, wherein the laser treatment device is operated at a maximum power density for the laser treatment device throughout the duration of supplying light energy to the area.

19. The method of claim 1, wherein the laser treatment device is operated at a maximum power density of 450 mw/cm².

20. The method of claim 1, wherein the capacitive resistive electric transfer therapy includes treating at least a portion of the area using a capacitive resistive electric transfer therapy device, and
wherein the capacitive resistive electric transfer therapy device is configured to operate in a capacitive mode and a resistive mode.

21. The method of claim 20, wherein the capacitive resistive electric transfer therapy includes treating at least the portion of the area using the capacitive mode followed by treating at least the portion of the area using the resistive mode.

22. The method of claim 20, wherein the capacitive resistive electric transfer therapy device is configured to supply capacitive and resistive radio frequency currents from 300 kHz to 1 MHz to at least a portion of the area.

23. The method of claim 1, wherein the shock wave therapy includes treating at least a portion of the area using a shock wave device.

24. The method of claim 23, wherein the shock wave device has a frequency of from 1 Hz to 12 Hz.

25. The method of claim 23, wherein a power and a frequency of the shock wave device is increased with each successive treatment session of the plurality of treatment sessions.

26. The method of claim 1, wherein the acoustical vibration therapy includes treating at least a portion of the area using an acoustical vibration device configured to supply a plurality of frequencies and a plurality of amplitudes of mechanical vibration to the portion of the area.

27. The method of claim 26, wherein the acoustical vibration device is configured to supply acoustical vibration having a frequency of 0-300 Hz and plurality of amplitudes.

28. The method of claim 26, wherein a frequency and an amplitude of the acoustical vibration supplied by the acoustical vibration device is increased with each successive treatment sessions of the plurality of treatment sessions.

29. The method of claim 1, wherein the plurality of treatment sessions is one of equal to and greater than five treatments.

30. The method of claim 1, wherein the plurality of treatment sessions is one of equal to and greater than ten treatments.

31. The method of claim 1, wherein the first predetermined number of treatment sessions is one of equal to and greater than five treatment sessions.

32. The method of claim 10, wherein the first predetermined number of treatment sessions is one of equal to and greater than five treatment sessions.

33. The method of claim 32, wherein the second predetermined number of treatment sessions is one of equal to and greater than five treatment sessions.

34. The method of claim 32, wherein the first predetermined number of treatment sessions is one of less than and equal to eight treatment sessions.

35. The method of claim 33, wherein the second predetermined number of treatment sessions is one of less than and equal to eight treatment sessions.

36. The method of claim 10, wherein the first predetermined number of treatment sessions is one of equal to and greater than fourteen treatment sessions.

37. The method of claim 36, wherein the second predetermined number of treatment sessions is one of equal to and greater than fourteen treatment sessions.

38. A method of treatment of musculoskeletal conditions, the method comprising the steps of:
treating an area of a body with a plurality of treatment sessions,
wherein each treatment session of the plurality of treatment sessions comprises a synergistic combination of sequentially administered therapeutic modalities administered to the area,
wherein, in a first subset of the plurality of treatment sessions, the therapeutic modalities comprise laser therapy followed by at least one of capacitive resistive electric transfer therapy, shock wave therapy, and acoustical vibration therapy,
wherein, in a second subset of the plurality of treatment sessions, the therapeutic modalities comprise laser therapy followed by at least one of capacitive resistive electric transfer therapy, shock wave therapy, and acoustical vibration therapy, and
wherein the therapeutic modalities of the first subset are different from the therapeutic modalities of the second subset.

39. A system for treatment of musculoskeletal conditions, the system comprising:
a laser therapy device;
a capacitive resistive electric transfer therapy device;
a shock wave therapy device; and
an acoustical vibration therapy device,
wherein the laser therapy device, the capacitive resistive electric transfer therapy device, the shock wave therapy device, and the acoustical vibration therapy device are configured to treat an area of a body with a plurality of treatment sessions, wherein each treatment session of the plurality of treatment sessions comprises sequentially administering therapeutic modalities to the area using at least two of the laser therapy device, the capacitive resistive electric transfer therapy device, the shock wave therapy device, and the acoustical vibration therapy device.
